(19) United States
(12) Patent Application Publication
(10) Pub. No.: US 2026/0212211 A1
(43) Pub. Date: Jul. 23, 2026

(54) METHOD, PROGRAM, AND DEVICE FOR CONSTRUCTING MODEL OPTIMIZED FOR ANALYSIS OF BIO SIGNALS

(71) Applicant: MEDICAL Al CO., LTD., Seoul (KR)

(72) Inventors: Joonmyoung KWON, Seoul (KR); Byeongtak LEE, Seoul (KR); Yongyeon JO, Seoul (KR)

(21) Appl. No.: 19/141,182

(22) PCT Filed: Jan. 11, 2024
(86) PCT No.: PCT/KR2024/000562

§ 371 Date: Jun. 19, 2025

(30) Foreign Application Priority Data
Jan. 12, 2023 (KR) 10-2023-0004793
Jan. 11, 2024 (KR) 10-2024-0004569

(51) Int. Cl. G06N 3/0985

(52) U.S. Cl. CPC G06N 3/0985

(57) ABSTRACT

According to embodiments of the present disclosure, there are disclosed a method, program and device for constructing a model optimized for the analysis of biosignals that are performed by a computing device. The method may include: determining the fundamental structure of a machine learning model based on user input; and performing hyperparameter tuning for constructing a machine learning model optimized for the analysis of biosignals based on the determined fundamental structure.

### TECHNICAL FIELD

The present disclosure relates to artificial intelligence technology in the medical field, and more particularly, to a hyperparameter optimization method for improving the performance of analysis of biosignals.

### BACKGROUND ART

Electrocardiograms record the electrical activities of the heart over time and are used to detect various heart-related diseases. Over the years, various machine learning models have been developed for electrocardiogram analysis. Preceding studies have explored the potential of electrocardiogram analysis using machine learning models, but no systematic study has been conducted on network architecture optimization. Network architecture optimization, which is the process of tuning hyperparameters, is considerably important for developing efficient and accurate models.

The optimal hyperparameters may vary depending on the task. Accordingly, it is important to tune them to achieve optimal performance for a specific task. The efficiency of hyperparameter optimization is highly influenced by a predefined search space. A large search space does not guarantee optimal performance, but rather may increase the time and resource cost, like in the case of finding a needle in a vast desert. Therefore, it is important to set an appropriate search space.

### DISCLOSURE

### Technical Problem

An object of the present disclosure is to provide a method for effectively defining a parameter search space and then performing optimal network scaling by reflecting therein the characteristics of biosignal analysis that are different from those of image analysis.

However, the objects to be achieved in the present disclosure are not limited to the object mentioned above, and other objects not mentioned may be clearly understood based on the following description.

### Technical Solution

According to one embodiment of the present disclosure for achieving the above-described object, there is disclosed a method of constructing a model optimized for the analysis of biosignals that is performed by a computing device including at least one processor. The method may include: determining the fundamental structure of a machine learning model based on user input; and performing hyperparameter tuning for constructing a machine learning model optimized for the analysis of biosignals based on the determined fundamental structure.

Alternatively, the performing hyperparameter tuning for constructing a machine learning model optimized for analysis of biosignals based on the determined fundamental structure may include: determining a search space of the scaling parameters based on a correlation between the performance of analysis of biosignals and scaling parameters; and applying a combination of scaling parameters selected within the determined search space to a machine learning model having the determined basic structure.

Alternatively, the scaling parameters may include layer depth, channels, and a kernel.

Alternatively, the performance of analysis of the biosignals and the layer depth may have a negative correlation.

Alternatively, the performance of analysis of the biosignals and the size of the kernel may have a negative correlation.

Alternatively, the determining a search space of the scaling parameters based on a correlation between performance of analysis of biosignals and scaling parameters may include: determining a second search space by sequentially removing larger values for the layer depth and the size of the kernel and a smaller value for the number of channels within a first search space according to the correlation between the performance of analysis of biosignals and the scaling parameters.

Alternatively, the threshold values that serve as criteria for the removal may be determined based on the characteristics of analysis of the biosignals.

Alternatively, the characteristics of analysis of the biosignals may be determined based on at least one of the types of the biosignals and the analysis classes of the biosignals.

According to one embodiment of the present disclosure for achieving the above-described object, there is disclosed a computer program that is stored in a computer-readable storage medium. The computer program may cause operations for constructing a model optimized for analysis of biosignals to be performed when executed by at least one processor. In this case, the operations may include operations of: determining the fundamental structure of a machine learning model based on user input; and performing hyperparameter tuning for constructing a machine learning model optimized for the analysis of biosignals based on the determined fundamental structure.

According to one embodiment of the present disclosure for achieving the above-described object, there is disclosed a computing device for constructing a model optimized for the analysis of biosignals. The computing device may include: a processor including at least one core; and memory including program codes executable by the processor. In this case, the processor may determine the fundamental structure of a machine learning model based on user input, and may perform hyperparameter tuning for constructing a machine learning model optimized for the analysis of biosignals based on the determined fundamental structure.

### Advantageous Effects

According to the method of the present disclosure, it may be possible to search for one or more scaling parameters that provide high performance for the analysis of biosignals by investing small amounts of time and cost.

In addition, by performing hyperparameter tuning through the found scaling parameters, a model optimized for the analysis of biosignals may be constructed and utilized for the analysis of biosignals.

### DESCRIPTION OF DRAWINGS

FIG. 1 is a block diagram of a computing device according to one embodiment of the present disclosure;

FIG. 2 shows the performance evaluation results of an electrocardiogram analysis model for individual combinations of scaling parameters;

FIG. 3 shows the correlation between electrocardiogram analysis classes and performance evaluation indices; and

FIG. 4 is a flowchart showing a method of constructing a model optimized for analyzing biosignals according to one embodiment of the present disclosure.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “acquisition” used herein can be understood to mean not only receiving data over a wired/wireless communication network connecting with an external device or a system, but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

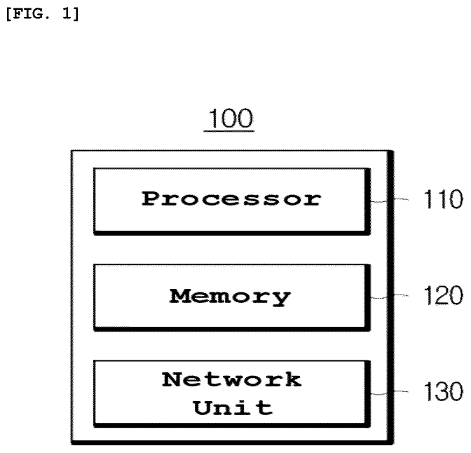

FIG. 1 is a block diagram of a computing device according to one embodiment of the present disclosure.

A computing device 100 according to one embodiment of the present disclosure may be a hardware device or a part of a hardware device that performs the comprehensive processing and computation of data, or may be a software-based computing environment connected over a communication network. For example, the computing device 100 may be a server that is a main agent for performing an intensive data processing function and sharing resources, or may be a client that shares resources through interaction with a server. Alternatively, the computing device 100 may be a cloud system in which multiple servers and clients comprehensively process data while interacting with each other. Since the above description is only one example related to the type of computing device 100, the type of computing device 100 may be configured in various manners within a range understandable to those skilled in the art based on the content of the present disclosure.

Referring to FIG. 1, the computing device 100 according to one embodiment of the present disclosure may include a processor 110, memory 120, and a network unit 130. However, FIG. 1 is only an example, and the computing device 100 may further include other components for implementing a computing environment. Furthermore, only some of the disclosed components may be included in the computing device 100.

The processor 110 according to one embodiment of the present disclosure may be understood as a constituent unit including hardware and/or software for performing computing operations. For example, the processor 110 may read a computer program and perform data processing for machine learning. The processor 110 may process operation processes such as the processing of input data for machine learning, the extraction of features for machine learning, and the computation of errors based on backpropagation. The processor 110 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 110 described above are only examples, the type of processor 110 may be configured in various manners within a range understandable to those skilled in the art based on the content of the present disclosure.

The processor 110 may construct a model for analyzing biosignals through hyperparameter optimization that reflects therein the analysis characteristics of biosignals. The processor 110 may determine the fundamental structure of a machine learning model based on user input. Furthermore, the processor 110 may perform hyperparameter tuning for the model having the determined fundamental structure. In this case, the hyperparameter tuning may be tuning for major scaling parameters that influence the scale and complexity of the model. For example, the processor 110 may determine the fundamental structure of a machine learning model to be a convolutional neural network based on user input. The processor 110 may tune scaling parameters such as the layer depth, channels, and kernel of a model whose fundamental structure is determined to be a convolutional neural network based on user input. In this case, the tuning of scaling parameters may be performed using a combination of selected scaling parameters within a search space determined based on the correlation between the performance of analysis of biosignals and the scaling parameters.

It is known that there is a positive correlation between the performance of the model and the scaling parameters in the field of general computer vision. However, tuning all scaling parameters to have the largest possible values as applied in the field of computer vision may not be viewed as being also applied to the field of biosignal analysis. Accordingly, the processor 110 may determine the search space of each of the scaling parameters based on the correlation between the performance of the model and each of the scaling parameters such as layer depth, channels, and a kernel according to the purpose of biosignal analysis. Then, the processor 110 may derive a combination of scaling parameters within the search space based on user input or by using a technique such as random search.

More specifically, the processor 110 may determine a second search space by sequentially removing larger values for the layer depth and the size of the kernel and a smaller value for the number of channels within a first search space according to the correlation between the performance of analysis of biosignals and the scaling parameters. The first search space may be understood as the initial search range that is commonly used in accordance with the fundamental structure of the model. The second search space may be understood as the search range that is determined for hyperparameter tuning for the construction of a model for analyzing biosignals. Since the layer depth and the size of the kernel have a negative correlation with the performance of analysis of biosignals, the processor 110 may remove values for the layer depth and the size of the kernel from larger values to threshold values within the first search space. In contrast, since the number of channels has a positive correlation with the performance of analysis of biosignals, the processor 110 may remove values for the number of channels from a smaller value to a threshold value within the first search space. In this case, the threshold values may each be determined for each of the scaling parameters based on the characteristics of analysis of biosignals. The characteristics of analysis of biosignals may be determined based on at least one of the types of biosignals and the analysis classes of biosignals. That is, the characteristics of analysis of biosignals may vary depending on whether the biosignals are an electrocardiogram or heart rates, and may vary depending on whether the class to be diagnosed through the analysis of biosignals is an arrhythmia or a myocardial infarction. When the characteristics of analysis of biosignals are determined based on at least one of the types of biosignals and the analysis classes of biosignals based on user input, the processor 110 may determine a threshold value matching the characteristics of analysis of biosignals for each scaling parameter based on a previously constructed database. Then, the processor 110 may extract the second search space from the first search space by using the determined threshold value.

That is, the processor 110 may determine a search space for hyperparameter tuning optimized for the analysis of biosignals by removing the space where the performance of analysis of biosignals decreases and extracting the space where the performance of analysis of biosignals increases for each of the scaling parameters in the search space that is commonly used in the field of artificial intelligence. Then, the processor 110 may extract an optimal combination within a search space and apply it to a model having a fundamental structure. Through this process, the processor 110 may construct a model optimized for the analysis of biosignals.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize and manage the data required for the processor 110 to perform operations, combinations of data, and the program codes executable by the processor 110. For example, the memory 120 may store the medical data received via the network unit 130 to be described later. The memory 120 may store the program codes that operate a machine learning model to receive medical data as input and perform learning, the program codes that operate a machine learning model to receive medical data as input and perform inference according to the purpose of use of the computing device 100, and the processed data that is generated as the program codes are executed.

The network unit 130 according to one embodiment of the present disclosure may be understood as a constituent unit that transmits and receives data via any type of known wired/wireless communication system. For example, the network unit 130 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 130 may be applied in various manners other than the above-described examples.

The network unit 130 may receive data, required for the processor 110 to perform operations, through wired/wireless communication with any system, any client, or the like. Furthermore, the network unit 130 may transmit data, generated through the operations of the processor 110, through wired/wireless communication with any system, any client, or the like. For example, the network unit 130 may receive medical data through communication with a database within a hospital environment, a cloud server that performs tasks such as the standardization of medical data, a client such as a smart watch, a medical computing device, or the like. The network unit 130 may transmit the output data of a machine learning model, and the intermediate data, processed data, and like derived from the computational process of the processor 110 through communication with the above-described database, server, client, or computing device.

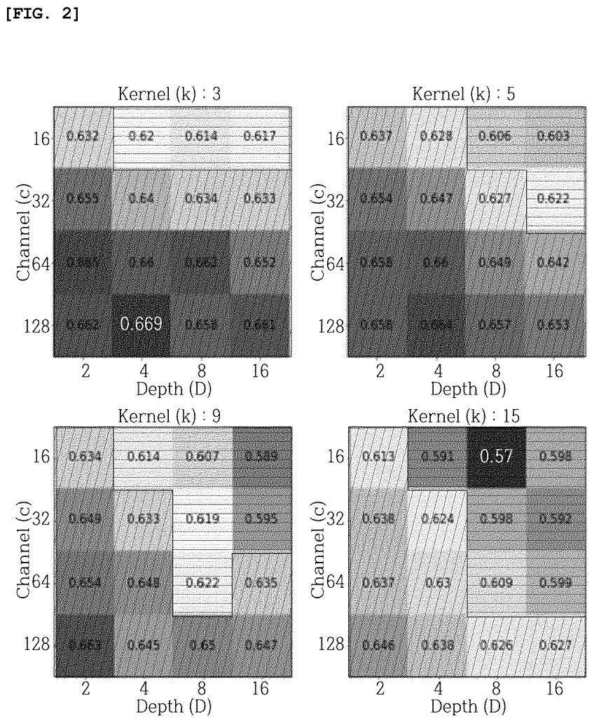

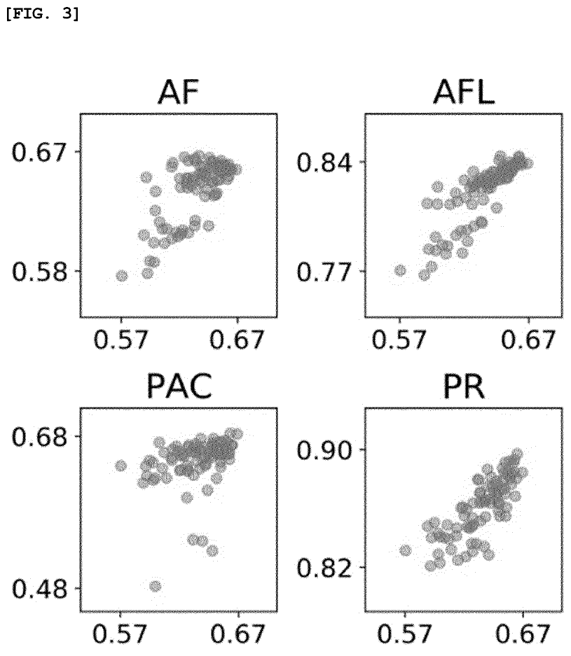

The experimental results on model extensions that may improve performance in electrocardiogram classification will be described below. Based on this, the correlations between the analysis performance of biosignals and scaling parameters will be discussed. FIG. 2 shows the performance evaluation results of an electrocardiogram classification model for individual combinations of scaling parameters. FIG. 3 shows the correlation between electrocardiogram classification classes and performance evaluation indices.

### 1. FUNDAMENTAL STRUCTURE OF MODEL

Equation 1 below represents the structure of a residual neural network.

$$y = ( FC ∘ GAP ∘ {R}_{4} ∘ {R}_{3} ∘ {R}_{2} ∘ {R}_{1} ∘ S ) ( x ) \qquad ( 1 )$$

In this equation, S denotes a single stem block, Rₙ denotes a residual block, GAP denotes a global average pooling layer, and FC denotes a fully connected layer. The stem block S is composed of a single unit function F together with a max pooling layer as shown in Equation 2. The function F is composed of a one-dimensional convolution, a batch normalization, and a ReLU activation function as shown in Equation 3.

$$S = Pool ( F ( x , w ) ) \qquad ( 2 )$$

$$F ( x , w ) = σ ( BN ( Conv ( x , w ) ) ) , w ∈ {R}^{kernel \times {C}_{in} \times {C}_{out}} \qquad ( 3 )$$

In the above equation, w denotes the convolution weight, which is defined as the product of the size of the kernel kernel, the number of input channels Cᵢₙ, and the number of output channels Cₒᵤₜ. Assuming that there are 12 leads in the electrocardiogram, the number of input channels for convolution is set to 12 (i.e., w∈Rkernel×12×Cₒᵤₜ).

The residual block R is composed of multiple residual layers L, as shown in Equation 4.

$$R = ( {L}_{d} ∘ … ∘ {L}_{1} ) ( x ) \qquad ( 4 )$$

In this equation, d is the layer depth. The structure of L may be represented by Equation 5, and is composed of two sequential functions F. In the first residual layer L, the first function F, excluding the initial residual block, doubles the number of output channels via convolution Conv. The subsequent F maintains the same number of input and output channels. The input x is skip-connected with the output after the second batch normalization layer and passes through the activation function. Before this skip connection, the initial L of Rₙ processes x by using Pool and Conv, and the remaining layers perform identity mapping.

$$L = \{ \begin{matrix}F ( ( F ( x , w ) ) , 2 w ) + Pool ( Conv ( x ) ) & if n > 1 , d = 1 \\ F ( ( F ( x , 2 w ) ) , 2 w ) + x & otherwise\end{matrix} \qquad ( 5 )$$

In this equation, n denotes the index of the block, and d denotes the index of the layer within the block.

### 2. SCALING PARAMETERS

The hyperparameters that influence the network scale are the depth of the layer (depth D), the number of convolution channels (channels C), and the size of the convolution kernel (kernel size K). To investigate the influences of these scaling parameters on performance, each search space is defined as described in Table 1. The search space is set to include various scaling parameters used for electrocardiogram classification. Since four residual blocks are used, D∈{2, 4, 8, 16} corresponds to 18, 34, 66, and 130 convolutional layers in total, and C∈{16, 32, 64, 128} corresponds to 128, 256, 512, and 1024 final output channels.

TABLE 1

| Scaling Parameter | Search Space |
|---|---|
| Depth D | {2, 4, 8, 16} |
| Kernel Size K | {3, 5, 9, 15} |
| Channel Number C | {16, 32, 64, 128} |

### 3. DATASET

For the experiment, the Physionet Challenge 2021 dataset was used. This dataset is composed of standard 12-lead electrocardiograms having cardiac arrhythmia labels, and the signal length is between 10 and 60 seconds. The total number of electrocardiograms is about 88,000, and each electrocardiogram is associated with one or more labels of 26 diagnostic classes.

### 4. EVALUATION

The averages of the F1 scores of classes were evaluated using the macro average F1 scores. The dataset was divided into training, validation, and test sets at ratios of 0.7, 0.15, and 0.15. In order to rigorously evaluate the influences of the scaling parameters, the models were trained with 50 hyperparameter combinations for the individual scaling parameters. Then, the models having exhibited the best performance were compared with each other.

### 5. RESULTS

### 5.1. Influence of Scaling Parameter Optimization on Electrocardiogram Classification

FIG. 2 shows the F1 scores of electrocardiogram classification for residual neural networks having different layer depths D, numbers of channels C, and kernel sizes K. The colors of the boxes represent performance. As the red color (in the hatched area) is darker, the performance becomes higher, whereas as the blue color (in the horizontal line area) is darker, the performance becomes lower.
Layer depth D: It can be seen that the performance consistently improved as the depth D decreased. This trend was exhibited independently of the kernel size K and the channel C. This result is in sharp contrast to that in the field of computer vision in which the performance becomes better as the network is deeper.
Number of channels C: It can be seen that there was a positive correlation between the number of channels C and the performance. In most cases, the performance generally improved as the number of channels C increased. This result is consistent with conventional knowledge in computer vision, and indicates that a wider network is also advantageous for the electrocardiogram classification task.
Kernel size K:

It can be seen that the left panels of FIG. 2 are noticeably redder than the right panels. This shows that the performance tends to deteriorate as the kernel size K increases, which is the result opposite to the result in the field of computer vision.

### 5.2. Effect of Diagnostic Class

FIG. 3 shows the F1 scores for respective classes based on the average F1 scores according to the network scale. The correlations of the results for four classes, including atrial fibrillation (AF), atrial flutter (AFL), atrial premature contraction (PAC), and sinus arrhythmia (SA), were found to be less than 0.7.

For AF and AFL, two clusters were formed. In both results, the depths of the lowest cluster were found to be 8 and 16, respectively. For PAC and SA, there were observed a few cases where the F1 scores for respective classes were significantly lower than the average F1 scores. These outliers are each associated with a depth of 1 and a kernel size of 3 or 5. These results suggest that the optimal scaling parameters vary across diagnostic classes, and that the selection of the depth and the kernel size may be particularly sensitive to class types.

### 6. CONCLUSION

The experimental results show that a model having a shallower network, a larger number of channels, and a smaller kernel size improved the performance of electrocardiogram classification. This result is different from the conventional knowledge in the field of computer vision. Furthermore, it shows that applying this result to a hyperparameter optimization process improves the performance of electrocardiogram classification.

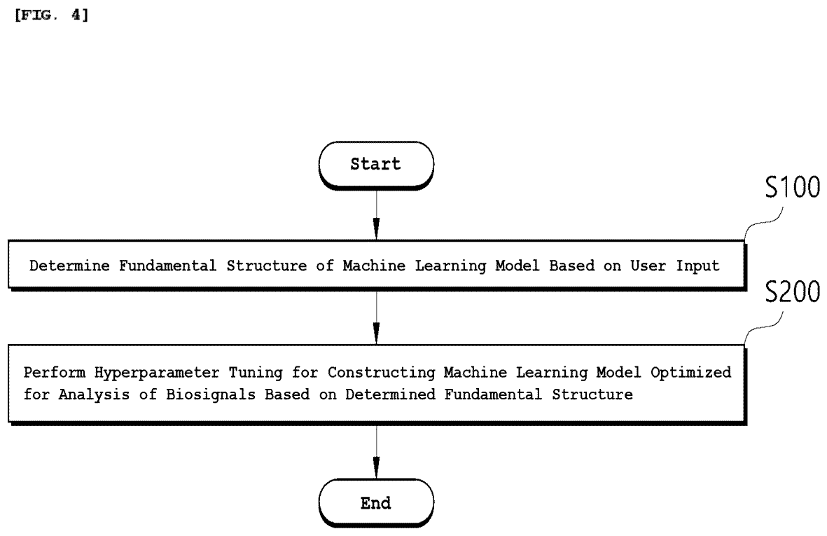

FIG. 4 is a flowchart showing a method of constructing a model optimized for analyzing biosignals according to one embodiment of the present disclosure.

Referring to FIG. 4, a computing device according to one embodiment of the present disclosure may determine the fundamental structure of a machine learning model based on a user input in step S100. For example, when the computing device is a server, the computing device may select the fundamental structure of a machine learning model based on a user command input via the interface of a client. A user may input a command by selecting one of the multiple fundamental structures listed via the interface of the client. The user may also input a command by selecting one of the templates prepared in accordance with the type or diagnostic class of a biosignal to be analyzed via the interface of the client. A method of inputting a command according to the present disclosure is not limited to the above-described example.

The computing device according to one embodiment of the present disclosure may perform hyperparameter tuning for constructing a machine learning model optimized for the analysis of biosignals based on the fundamental structure determined via step S100. The computing device may determine the search space of scaling parameters based on the correlation between the analysis performance of biosignals and the scaling parameters. The computing device may apply a combination of scaling parameters selected within the determined search space to the machine learning model having the fundamental structure determined via step S100. More specifically, the computing device may determine the search space of scaling parameters by sequentially removing larger values for the layer depth and the size of the kernel and a smaller value for the number of channels within the initial search space based on the correlation between the analysis performance of biosignals and the scaling parameters. In this case, the initial search space may be a search space that is commonly used for hyperparameter tuning in the field of artificial intelligence. Meanwhile, the computing device may use the analysis characteristics of biosignals determined based on at least one of the types of biosignals and the analysis classes of biosignals in order to determine the search space. For example, when the user sets the construction of a model for diagnosing arrhythmia through electrocardiogram analysis, the computing device may determine a threshold value, which is a criterion for determining the search space for each of the layer depth, the number of channels, and the size of the kernel by using the characteristics of electrocardiogram and arrhythmia. In this case, the determination of the threshold value may be performed by matching the analysis characteristics of biosignals with the threshold value for each scaling parameter and utilizing a previously constructed database. The computing device may determine the search space based on the threshold value for each scaling parameter and the correlation between the performance of the model and the parameters. Then, the computing device may select a combination of scaling parameters from the determined search space and apply it to the machine learning model having the fundamental structure. In this case, the selection of the combination may be performed based on user input or automatically by using a search technique such as random search.

The various embodiments of the present disclosure described above may be combined with one or more additional changed within the range embodiments, and may be understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A method of constructing a model optimized for analysis of biosignals, the method being performed by a computing device including at least one processor, the method comprising:
determining a fundamental structure of a machine learning model based on user input; and
performing hyperparameter tuning for constructing a machine learning model optimized for analysis of biosignals based on the determined fundamental structure.

2. The method of claim 1, wherein the performing hyperparameter tuning for constructing a machine learning model optimized for analysis of biosignals based on the determined fundamental structure comprises:
determining a search space of the scaling parameters based on a correlation between performance of analysis of biosignals and scaling parameters; and
applying a combination of scaling parameters selected within the determined search space to a machine learning model having the determined basic structure.

3. The method of claim 2, wherein the scaling parameters comprise layer depth, channels, and kernel.

4. The method of claim 3, wherein the performance of analysis of the biosignals and the layer depth have a negative correlation.

5. The method of claim 3, wherein performance of analysis of the biosignals and a size of the kernel have a negative correlation.

6. The method of claim 3, wherein the determining a search space of the scaling parameters based on a correlation between performance of analysis of biosignals and scaling parameters comprises:
determining a second search space by sequentially removing larger values for the layer depth and a size of the kernel and a smaller value for a number of channels within a first search space according to the correlation between the performance of analysis of biosignals and the scaling parameters.

7. The method of claim 6, wherein the threshold values that serve as criteria for the removal are determined based on characteristics of analysis of the biosignals.

8. The method of claim 7, wherein the characteristics of analysis of the biosignals are determined based on at least one of types of the biosignals and analysis classes of the biosignals.

9. A computer program stored in a computer-readable storage medium, the computer program causing operations for constructing a model optimized for analysis of biosignals to be performed when executed by at least one processor, wherein the operations comprise operations of:
determining a fundamental structure of a machine learning model based on user input; and
performing hyperparameter tuning for constructing a machine learning model optimized for analysis of biosignals based on the determined fundamental structure.

10. A computing device for constructing a model optimized for analysis of biosignals, the computing device comprising:
a processor including at least one core; and
memory including program codes executable by the processor;
wherein the processor:
determines a fundamental structure of a machine learning model based on user input; and
performs hyperparameter tuning for constructing a machine learning model optimized for analysis of biosignals based on the determined fundamental structure.
